(19) United States
(12) Patent Application Publication
(10) Pub. No.: US 2026/0053491 A1
(43) Pub. Date: Feb. 26, 2026

(54) Self-Punching Soft Anchor Inserter

(71) Applicant: Arthrex, Inc., Naples, FL (US)

(72) Inventors: Peter J. DREYFUSS, Naples, FL (US); Neal S. ELATTRACHE, Las Angeles, CA (US); Asheesh BEDI, Ann Arbor, MI (US)

(73) Assignee: Arthrex, Inc., Naples, FL (US)

(21) Appl. No.: 19/269,386

(22) Filed: Jul. 15, 2025

Related U.S. Application Data
(60) Provisional application No. 63/686,943, filed on Aug. 26, 2024.

(51) Int. Cl. A61B 17/04; A61B 17/00

(52) U.S. Cl. CPC A61B 17/0401; A61B 2017/00004; A61B 2017/0404; A61B 2017/0406; A61B 2017/0409; A61B 2017/0414; A61B 2017/0464

(57) ABSTRACT

Surgical constructs, assemblies, kits and methods of tissue fixation are disclosed. A surgical inserter is a soft anchor inserter provided with a distal fork that engages a self-punching tip. The surgical instrument is configured to insert a soft anchor into tissue while the self-punching tip creates a pilot hole without fracturing the bone and without catching soft tissue when inserted percutaneously.

### CROSS REFERENCE TO RELATED APPLICATIONS

This application claim priority to US Provisional Application No. 63/686,943 filed on Aug. 26, 2024, the disclosure of which is hereby incorporated by reference in its entirety herein.

### BACKGROUND

The present disclosure relates to the field of surgery and, more particularly, to surgical instruments and methods of surgical repairs.

### SUMMARY

Surgical instruments, assemblies, and methods are disclosed. A surgical instrument is a soft anchor inserter provided with a distal fork that engages a self-punching tip. The instrument is configured to insert a soft anchor into tissue while the self-punching tip creates a pilot hole without fracturing the bone and without catching soft tissue when inserted percutaneously.

Methods of surgeries are also disclosed. A method of arthroscopic surgery can be conducted with a self-punching inserter designed to securely form a pilot hole and insert a soft anchor within the pilot hole. The method provides increased and stable bone penetration and eliminates catching of soft tissue when the inserter is passed percutaneously.

### BRIEF DESCRIPTION OF THE DRAWINGS

FIG. 1 illustrates a perspective view of a distal end of a surgical instrument in an unassembled state.

FIG. 2 illustrates a perspective view of a distal end of a surgical instrument in an assembled state.

FIG. 3 illustrates a top view of a distal end of a surgical instrument.

FIG. 4 illustrates a side view of the surgical instrument of FIG. 3.

### DETAILED DESCRIPTION

The present disclosure provides surgical instruments, assemblies, kits, and methods of surgery.

An inserter can be a soft anchor inserter. An inserter can be a self-punching soft anchor inserter. A self-punching soft anchor inserter has a geometry that is designed for inserting soft anchors and that creates a pilot hole for the soft anchor without fracturing the bone while eliminating the issue of catching soft tissue when inserted percutaneously. A self-punching soft anchor inserter can create a pilot hole while simultaneously inserting a fixation device (a soft anchor) within the pilot hole.

In an embodiment, a self-punching tip is assembled to a soft anchor inserter. The tip creates a punch tip for insertion. The tip can have a conical configuration. The tip can be provided with an eyelet to allow one or more additional sutures to be included in the repair (the retention suture that is through the eyelet). The tip can be made from metals and/or metal alloys (such as titanium or PEEK), or combinations thereof. The tip can be preferably made from a resorbable material. The tip can include magnesium and PGA/PLLA. The tip can be easily and securely attached to/assembled onto the inserter fork, to secure the soft anchor between the fork and the tip while simultaneously providing self-punching capabilities. The tip can be easily and securely detached from the inserter fork. The tip can remain in the pilot hole to secure the soft anchor within the pilot hole and increase the strength of the repair.

A surgical inserter can provide soft anchor insertion and bone hole formation without fracturing the bone and without soft tissue catching.

Methods of endoscopic or arthroscopic surgeries are also disclosed. An exemplary method includes inter alia the steps of: (i) employing a surgical inserter to position and hold a soft anchor within a fork of the surgical inserter; and (ii) forming a hole, socket or opening in tissue with a tip of the surgical inserter while the soft anchor is held within the inserter fork. The method can further include (iii) inserting the soft anchor into the hole, socket or opening formed in the tissue. The method can further include (vi) leaving the inserter tip within the hole, socket or opening formed in the tissue prior to step (iii).

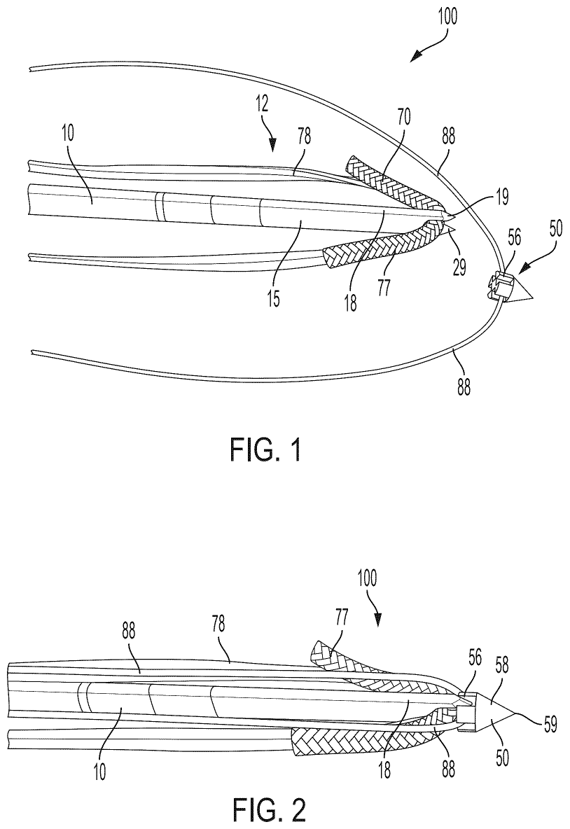

Referring now to the drawings, where like elements are designated by like reference numerals, FIGS. 1-4 illustrate exemplary surgical inserter 100 (device 100; self-punching soft anchor inserter 100; inserter assembly 100).

Surgical inserter 100 described below can be employed, for example, as part of a surgical procedure directed at repairing soft tissue injuries (e.g., rotator cuff injuries). To that end, surgical inserter 100 is configured to penetrate hard tissue (such as bone) to form a hole (for example, a pilot hole) and to insert a soft anchor within the formed hole. Surgical inserter 100 can be used alone, or in combination with additional surgical instruments such as additional penetrators and/or suture passers/retrieval instruments as part of various surgical guide assemblies and kits.

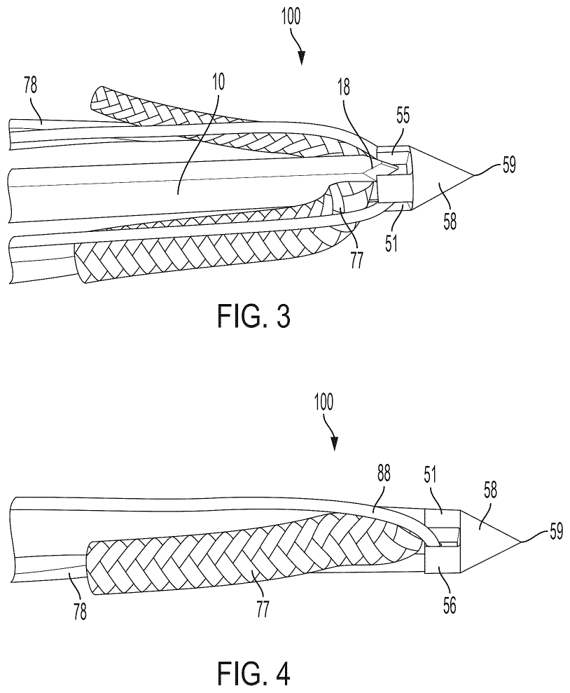

FIG. 1 illustrates an unassembled view of surgical inserter 100. FIG. 2 illustrates an assembled view of surgical inserter 100. FIGS. 3 and 4 illustrate additional views of surgical inserter 100.

Surgical inserter 100 of FIG. 1 comprises a shaft or tube 10 connected to a handle (not shown) at a proximal end 11 of the shaft. A tip 50 securely and removably attaches to the shaft 10 at a distal end 12 of the shaft 10.

Shaft 10 has an elongated body with a longitudinal axis 10a, proximal end 11 (attached to the handle) and distal end 12 configured to securely attach to/detach from the tip 50. Shaft 10 can be formed integral with the handle (forms a unitary structure). Shaft 10 can be rigid or partially rigid and can be formed of various materials such as metals, alloys, polymers, plastics, or combinations thereof. Shaft 10 can have various cross-sections such as circular or elliptical, among many others. The shaft body can be constructed with enough strength and rigidity to withstand being forcibly advanced through soft tissue and bone during a surgical procedure.

As shown in FIG. 1, shaft 10 can be provided with two slanted, non-vertical, angled faces 15 at its most distal end 12. Each of the angled faces 15 terminates in a respective prong 18 (spike 18; tine 18; elongated member 18) having a most distal end 19 terminating in a sharp point 19. Prongs 18 and sharp points 19 form a forked opening 29 (forked distal end 29; fork 29; recess cavity 29; aperture 29; slot 29; gap 29; slit 29; cut 29).

In an exemplary-only embodiment, the slanted, angled faces 15 are about parallel to each other and each form an angle of about 45 degrees relative to longitudinal axis 10a of the shaft 10. However, the slanted, non-vertical, angled faces 15 can be slanted at any angles as long as the faces are fully complementary.

Forked opening 29 is an elongated opening extending along the longitudinal axis 10a of the shaft 10 and surgical inserter 100. Forked opening 29 has a length L1, a width W1, and an overall geometry that can be similar to that of a semicircle. Forked opening 29 extends between the two prongs 18 terminating in sharp points 19.

Forked opening 29 engages and securely retains fixation device 70. In an embodiment, fixation device 70 is a soft anchor 70 (an all-suture soft anchor 70). In an embodiment, forked opening 29 retains a soft anchor sheath 77 or tubular member 77 of soft anchor 70. In an embodiment, the dimensions of the forked opening 29 (length L1 and width W1) are about similar to the cross-sectional dimensions of the soft anchor sheath 77 of the soft anchor 70. In an embodiment, forked opening 29 allows secure engagement and retention of anchor sheath 77, and as described below. The forked opening 29 can be dimensioned to allow easy, secure engagement of one or more anchor sheaths therethrough.

Tip 50 is illustrated in FIG. 1 in the unassembled state. In an embodiment, tip 50 is a self-punching tip designed to be assembled to shaft 10 to form soft anchor inserter 100. The tip 50 creates a punch tip for insertion. The tip 50 can be a cone. The tip 50 can have a body 51 and a most distal end 58 with a conical configuration (conical end 58) that terminates in a sharp point 59 (sharp penetrating cutting point 59).

In an embodiment, body 51 of tip 50 is provided with a plurality of recesses and/or openings and/or slots 55 configured to engage and secure prongs 18 of forked opening 29. In an embodiment, body 51 of tip 50 is also provided with an eyelet 56 to allow one or more additional flexible strands 88 (for example, one or more sutures 88) to be included in the repair (such as a retention suture 88 that extends through the eyelet 56). The eyelet 56 can be located within the body 51 of tip 50 and in a direction about transversal to longitudinal axis 10a of shaft 10 of the inserter 100. The eyelet 56 can extend from one outer surface of the body 51 to another outer surface of the body 51, for example, from one outer surface to an opposite outer surface of the body 51 of tip 50. The eyelet 56 is dimensioned to allow easy passage and sliding of at least one flexible strand 88 through it.

The tip 50 can be made from metals and/or metal alloys (such as titanium, titanium alloys, or PEEK), or combinations thereof. The tip 50 can be preferably made from a resorbable material. The tip 50 can be preferably made from a bioabsorbable, bioresorbable, or partially absorbable material. The tip 50 can include magnesium and/or magnesium alloy and PGA/PLLA. The tip 50 can be easily and securely attached to (assembled onto) the inserter fork 29, to secure the soft anchor 70 between the fork 29 and the tip 50, while simultaneously providing self-punching capabilities. The tip 50 can act both as a cutting instrument and implant.

When assembled with tip 50, each prong 18 of shaft 10 abuts a complementary face 55a of slot 55 of tip 50 to allow secure retention of the soft anchor 70 while the tip 50 is advanced into tissue (for example, bone) to form a pilot hole (such as a bone opening, socket, or hole) with conical end 58 and sharp cutting point 59, and to allow for insertion of the soft anchor 70 within the pilot hole. Insertion of soft anchor 70 within the pilot hole is conducted simultaneously with the formation of the pilot hole by the conical tip 50. Soft anchor sheath 77 and tip 50 are inserted within the pilot hole and secured therein. Flexible strand(s) 78 from the soft anchor 70 can be further employed for additional surgical procedures, as necessary and as desired.

Soft anchor 70 can be a fixation device in the form of an all-suture soft anchor 70 (soft suture anchor) provided with a soft anchor sleeve 77 (sheath 77; tubular member 77) with two open ends and one or more flexible shuttling strands 78 extending through the soft anchor sleeve 77 (sheath). Flexible strands 78 can extend through the sleeve 77 in similar or different directions and/or orientations and/or locations. The flexible sleeve 77 with the one or more shuttling strands 78 is secured into or onto bone (into the pilot hole formed with tip 50 of inserter 100), and the strands 78 allow passing of additional flexible strands such as sutures or tapes to pass over soft tissue and be secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,”the disclosure of which is incorporated by reference in its entirety herein.

A surgical inserter 100 comprises: a shaft 10 having a proximal end 11, a distal end 12, and a longitudinal axis 10a; a forked opening 29 provided at the distal end 12; and a tip 50 configured to engage the forked opening 29. The forked end 29 retains a fixation device 70. The tip 50 is provided with a conical end 58 terminating in a sharp cutting point 59 to form a pilot hole. Once the pilot hole has been formed, sheath 77 with at least one flexible strand 78 of soft anchor 70 are positioned and secured (with the tip 50) within the pilot hole formed by the tip 50. The tip 50 is preferably resorbable, to allow the tip to be left within the pilot hole and provide increased interference with the sheath 77 of the soft anchor 70. Body 51 of tip 50 can further include at least one eyelet 56 to allow one or more flexible strands 88 to slidably and removably pass through the at least one eyelet 56. In an embodiment, a longitudinal axis 10a of shaft 10 is about parallel and concentric (coincides) with a longitudinal axis of the tip 50. In an embodiment, the forked opening 29 matingly engages side slots 55 provided within body 51 of tip 50.

Methods of endoscopic or arthroscopic surgeries are also disclosed.

An exemplary method includes inter alia the steps of: (i) employing a surgical inserter 100 to position and hold a fixation device 70 within a forked slot 29 of the surgical inserter 100; and (ii) forming a pilot hole with tip 50 of the surgical inserter. The method can further include conducting steps (i) and (ii) simultaneously. The method can further include: (iii) securing both the tip 50 and the fixation device 70 within the pilot hole; and (iv) removing the inserter shaft 10. The method can further include the step of conducting at least one surgical procedure with flexible couplers 78, 88. The at least one surgical procedure can be a soft tissue repair such as rotator cuff repair.

Another exemplary method includes inter alia the steps of: (i) securing a soft sheath or tubular member 77 of a soft anchor 70 into a forked opening 29 of an inserter shaft 10; (ii) matingly attaching an inserter tip 50 to the forked opening 29 to form an assembled inserter 100; (iii) forming a pilot hole (opening or socket) within hard tissue such as bone with the inserter tip 50; (iv) leaving the soft anchor 70 and the inserter tip 50 within the pilot hole; (v) removing the inserter shaft 10; and (vi) securing at least one of flexible strand(s) 78 of the soft anchor 70 to additional structures (such as, for example, additional fixation devices and/or tissue such as soft tissue or bone). The additional fixation devices can be knotted or knotless anchors. At least one of the additional fixation devices can be a hard body anchor, a soft body anchor, or an all-suture soft anchor. At least one additional fixation device can be a knotless anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein.

The self-punching soft anchor inserter 100 has a geometry designed for inserting soft anchors 70 while creating a pilot hole for the soft anchor 70 (without fracturing the bone and without the issue of catching soft tissue when inserted percutaneously). A self-punching tip 50 is assembled to shaft 10 of a soft anchor inserter 100. The inserter tip 50 easily passes through the soft tissue (without catching any soft tissue) and stays within the formed pilot hole. In this manner, soft anchor 70 secured within the pilot hole and reinforced by inserter tip 50 (which is left within the pilot hole) becomes and works more like a hard anchor rather than a soft anchor.

Surgical inserter 100 detailed above has applicability to various open or arthroscopic procedures including procedures for re-approximating bone to bone or soft tissue to bone, for example, shoulder rotator cuff repairs, capsulolabral reconstruction, SLAP repairs, as well as ankle, knee, elbow or foot repairs, among many others.

Surgical inserter 100 detailed above can be part of a surgical kit, assembly, or system to simplify the surgeon's task of selecting a specific instrument and to aid in the overall surgical procedure. Surgical inserter 100 can be part of an assembly that includes additional instruments such as suture passers, cutting devices, fixation devices (such as anchors, screws, suture-button constructs, plates, etc.) and flexible members (such as flexible couplers, sutures, suture tapes, strands, etc.) to be employed in conjunction with the tissue repairs conducted with surgical inserter 100. Surgical inserter 100 can be reusable or disposable.

The flexible strands 78, 88 can be formed of any flexible material such as suture or tape, or combination of suture and tape. The suture can be in the form of any known suture construct, such as multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). The tape can be formed of suture tape, for example, Arthrex FiberTape®, which is a high strength suture tape that is braided and rectangular-like in cross section and as disclosed in U.S. Pat. No. 7,892,256, the disclosure of which is hereby incorporated by reference in its entirety herein. Flexible strands 78, 88 can include any soft, flexible strand of material. Flexible strands 78, 88 can consist essentially of elastic suture.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A surgical inserter comprising:
a shaft with a longitudinal axis, a proximal end, and a distal end;
a forked opening provided at the distal end of the shaft;
a fixation device provided within the forked opening; and
an inserter tip removably attached to the forked opening of the shaft.

2. The surgical inserter of claim 1, wherein the forked opening is configured to capture and retain the fixation device, and to mattingly engage the inserter tip.

3. The surgical inserter of claim 1, wherein the inserter tip has a conical configuration for forming a pilot hole within tissue.

4. The surgical inserter of claim 3, wherein the forked opening is configured to allow the fixation device to be freed and secured within the pilot hole as the surgical inserter is removed from the tissue.

5. The surgical inserter of claim 1, wherein the inserter tip includes a bioabsorbable material.

6. The surgical inserter of claim 1, wherein the inserter tip consists essentially of a bioresorbable material.

7. The surgical inserter of claim 1, wherein the inserter tip includes at least one transverse eyelet to allow one or more flexible strands to slidingly and removably pass therethrough.

8. The surgical inserter of claim 1, wherein the forked opening is formed by two symmetrical prongs configured to engage corresponding slots formed within an outer surface of the inserter tip.

9. The surgical inserter of claim 1, wherein the longitudinal axis of the shaft coincides with a longitudinal axis of the inserter tip.

10. The surgical inserter of claim 1, wherein the fixation device is a soft suture anchor, the tissue is bone, and wherein flexible strands attached to a sheath of the soft suture anchor are further secured to another fixation device or another tissue.

11. The surgical inserter of claim 10, wherein the another fixation device is an anchor, a button, a screw, or a plate.

12. The surgical inserter of claim 1, wherein the surgical inserter is used in arthroscopic or endoscopic surgery.

13. The surgical inserter of claim 1, wherein the surgical inserter is used in rotator cuff reconstruction.

14. A method of surgery, comprising:
positioning a soft anchor within a forked opening of an inserter shaft;
attaching an inserter tip to the forked opening of the inserter shaft;
forming a pilot hole within bone with the inserter tip; and
removing the inserter shaft from the bone to leave both the soft anchor and the inserter tip within the pilot hole.

15. The method of claim 14, further comprising the steps of:
attaching at least one flexible strand from the soft anchor to soft tissue; and
approximating the soft tissue to the bone.

16. The method of claim 15, wherein the at least one flexible strand is suture or suture tape.

17. The method of claim 15, wherein the at least one flexible strand includes a loop.

18. The method of claim 15, wherein the soft tissue is tendon, ligament, or graft.

19. The method of claim 15, wherein the soft tissue is part of a rotator cuff repair.

20. The method of claim 15, wherein the inserter tip is formed of at least one bioabsorbable material.
